(19) United States
(12) Patent Application Publication
(10) Pub. No.: US 2012/0076774 A1
(43) Pub. Date: Mar. 29, 2012

(54) DETECTION AND/OR TREATMENT OF DISEASES ASSOCIATED WITH AUTOANTIBODIES

(76) Inventors: Philip L. Riches, Edinburgh (GB); Stuart H. Ralston, Edinburgh (GB); William D. Fraser, Liverpool (GB)

(21) Appl. No.: 13/263,274

(22) PCT Filed: Apr. 7, 2010
(86) PCT No.: PCT/GB10/00699

§ 371 Date: Dec. 16, 2011

Related U.S. Application Data
(60) Provisional application No. 61/180,516, filed on May 22, 2009.

(30) Foreign Application Priority Data
Apr. 7, 2009 (GB) 0905973.4

(51) Int. Cl. A61K 39/395; G01N 33/566; A61K 38/23; A61K 31/675; A61K 31/663; A61P 25/00; A61P 19/04; A61P 19/10; A61P 9/00; A61P 37/02; A61P 19/02; A61P 1/00; A61K 38/19; C07K 14/715

(52) U.S. Cl. 424/131.1; 424/184.1; 435/7.1; 435/7.93; 435/7.94; 436/501; 514/11.9; 514/89; 514/94; 514/114; 530/350

(57) ABSTRACT

The present invention provides a method for detecting autoantibodies to osteoprotegerin (OPG). The method comprises the step of providing a biological sample from a subject with, or at risk of osteoporosis and detecting whether or not any antibodies against osteoprotegerin (OPG) are present in said sample. In addition the invention provides methods useful in aiding the diagnosis/prognosis and/or therapeutic regimen for autoimmune and/or vascular disease in general.

### FIELD OF THE INVENTION

The present invention relates to methods of detecting autoantibodies to osteoprotegerin, as well as potential therapies for diseases associated with autoantibodies against osteoprotegerin.

### BACKGROUND TO THE INVENTION

Osteoporosis is a common disease associated with reduced bone mass and an increased risk of fragility fractures. Osteoporosis is a recognised complication of celiac disease but this is generally considered to be secondary to the effects of calcium and vitamin D deficiency and malabsorption.

The receptor activator of nuclear factor kappa B (RANK) signalling pathway plays a critical role in regulating bone mass and bone turnover (1). RANK is a member of the TNF receptor superfamily which is expressed on osteoclast precursors and dendritic cells. Activation of RANK signalling occurs on binding of RANKL which is a member of the TNF superfamily that is expressed by bone marrow stromal cells. This causes activation of several intracellular signalling pathways which promote osteoclastogenesis. The RANKL-RANK interaction is blocked by osteoprotegerin (OPG) which inhibits bone resorption by acting as a decoy receptor for RANKL. The importance of this pathway in osteoclastogenesis in man is emphasised by the fact that inherited deficiency of RANK or RANKL causes osteopetrosis (9) whereas loss of function mutations of OPG cause a generalised increase in bone turnover leading to bone deformity, osteoporosis, and fragility fractures in both mice and humans (2-4). Recent studies indicate that common polymorphisms of OPG also play a role in the genetic regulation of both Paget's disease of bone and osteoporosis (5-8). Conflicting evidence exists regarding the relevance of serum OPG levels in osteoporosis and specifically there have been no previous reports of bone disease caused by an acquired abnormality in OPG production or activity.

The present invention is based on the identification of autoantibodies to osteoprotegerin in a subject with severe high turnover osteoporosis.

### SUMMARY OF THE INVENTION

In a first aspect there is provided a method for detecting autoantibodies to osteoprotegerin (OPG), the method comprising the step of providing a biological sample from a subject with, or at risk of, osteoporosis and detecting whether or not any antibodies against osteoprotegerin (OPG) are present in the biological sample.

Autoantibodies of the present invention are understood to be antibodies which have been produced by a host's, especially human, immune system directed against OPG or an antigenic fragment thereof.

As mentioned above, the present invention is based on the identification of autoantibodies to OPG in a subject with severe high turnover osteoporosis and as such, the method of detection may be employed in aiding diagnosis of osteoporosis. The method of detection may not be used alone and may be used in conjunction with other tests, such as dual-x-ray absorptiometry. Nevertheless such a method, if positive, may facilitate in identifying a complicating factor for the osteoporosis and aid a clinician in deciding on a particular course of therapy.

However, in addition to the subject displaying osteoporosis, it was observed that the subject was also suffering from autoimmune celiac disease. Although it is known in the art that celiac disease can be associated with osteoporosis this is thought to be due to malabsorption of calcium, vitamin D and other nutrients. Without wishing to be bound by theory, the present inventors postulate that autoantibodies to OPG may not just be associated with osteoporosis, but may be a marker/associated with autoimmune conditions in general, including celiac disease, rheumatoid arthritis and inflammatory bowel disease which are often complicated by osteoporosis, but hitherto there has been no identification of autoantibodies to OPG in these conditions.

In addition, the inventors have observed that autoantibodies to OPG are present in patients with autoimmune hypothyroidism, rheumatoid arthritis, severe idiopathic osteoporosis and multiple sclerosis. Thus, without wishing to be bound by theory, it is postulated that autoantibodies to OPG may be associated with, or markers of, other conditions such as autoimmune hypothyroidism, rheumatoid arthritis, severe idiopathic osteoporosis and multiple sclerosis. As such, it should be understood that the term “autoimmune disease(s)” or autoimmune condition(s)” as used herein, relate to all the diseases and/or conditions described herein, including for example, celiac disease, autoimmune hypothyroidism, rheumatoid arthritis, and multiple sclerosis as well as other autoimmune disease such as, for example, SLE, scleroderma, connective tissue disease and/or other disorders of the immune system. Whilst osteoporosis is not generally considered to be an autoimmune disease, the observation that autoantibodies to OPG are present in patients with severe osteoporosis, suggests that in some cases osteoporosis might be considered an “autoimmune” disease where the autoimmune reaction is primarily directed against the skeleton and more specifically, to the OPG protein.”

Moreover, it is also known that osteoporosis and vascular disease often coexist and there is evidence to suggest that deficiency of OPG may contribute to the pathogenesis of vascular calcification. With the identification, for the first time, of autoantibodies to OPG, the present invention also provides a potential method of aiding in the diagnosis and/or treatment of vascular disease, which may be associated with OPG levels/function.

Without wishing to be bound by theory, it will be appreciated that autoantibodies to OPG will bind to OPG present in a subject thereby disrupting the natural activity of OPG, which in turn will affect the RANKL/RANK/OPG system/balance leading to downstream consequences which may be associated with autoimmune and/or vascular conditions.

Thus, the method of detection may be used in aiding the diagnosis/prognosis and/or therapeutic regimen for autoimmune and/or vascular disease in general. In one embodiment the detection methods disclosed herein, may be used to, for example, diagnose/evaluate and/or determine an appropriate therapeutic regimen for celiac disease, autoimmune hypothyroidism, rheumatoid arthritis, severe idiopathic osteoporosis and multiple sclerosis.

The biological sample may be any suitable sample, including blood, such as serum, plasma and the like, as well as urine, saliva and leukocytes isolated from blood.

The methods for the determination of said autoantibodies in a biological sample may be any known immunodiagnostic method which may be used for detecting antibodies. Examples include radioimmunoassay, ELISAs, sandwich assays and the like. A general description of suitable assays may be found in Immunoassay: A practical guide by Brian Law, CRC Press, 1996 and Immunoassay: A practical approach by James P. Gosling, Oxford University Press, 2000, which is hereby incorporated by way of reference.

One exemplary way in which such an immunodiagnostic method may be carried out would be to provide a substrate, such as a well of a microtitre plate, coated with purified OPG or fragments thereof capable of binding to OPG autoantibodies. Any anti-OPG present in a biological sample would be allowed to bind to this before washing away unreacted material. This bound anti-OPG in turn may be detected by way of a further, optionally labelled anti-anti-OPG antibody. However, a further antibody may not in fact be required and bound anti-OPG antibody may be directed by physical, electrophysical, or even spectrophotometric means, such as by Raman spectroscopy.

In another embodiment, a displacement or competitive binding assay may be provided. OPG is a decoy receptor for RANKL which binds RANKL and prevents it binding to RANK on the surface of osteoclasts and other target cells. Many RANKL assays take advantage of the fact that OPG can bind RANKL and these assays use OPG as a substrate on ELISA plates as a way of “capturing” RANKL present in serum or plasma samples. However if antibodies to OPG were present in serum, this would interfere with the ability of RANKL to bind OPG. This would be expected to give unusually low serum levels of RANKL in patients who have OPG antibodies, with an increase in RANKL as the serum samples are diluted. Alternatively, addition of serum from a patient with OPG antibodies to a sample containing a known amount of RANKL would cause the measured level to decrease. A control/negative control could be set up with known amounts of RANKL and OPG in order to give a baseline value. Moreover, if a sample is added which contains anti-OPG antibodies, this would bind to the available OPG in the sample, preventing its action of binding RANKL, which in turn will increase the amount of RANKL binding to RANK and this may also be detected.

The methods may also be carried out using known chip based technology or as a rapid (point-of-care) type assay.

The OPG may be human or animal and purified accordingly or may, for example, be produced by recombinant means and expressed genetically, in known prokaryotic or eukaryotic expression systems (see for example Sambrook and Russell: “Molecular Cloning: A Laboratory Manual”; 2001, CSHL Press,) Antigenic fractions of OPG may also be used. Once purified, the OPG or antigenic fraction thereof, may be used to generate a supply of antibodies including monoclonal antibodies, according to known methods and which may be used in the methods described herein. These antibodies may be labelled by radio, fluorescent, enzyme tags or any other means known in the art.

As well as the methods of detection described herein, the present invention also provides possible therapies for diseases associated with an imbalance of RANKL/RANK/OPG pathway, as described herein, as well as the downstream signalling cascade following RANK activation. In particular the diseases include osteoporosis, autoimmune disease (for example celiac disease, autoimmune hypothyroidism and severe idiopathic osteoporosis) and vascular disease. It is envisaged that the therapies may aim to deactivate (block) or remove the antibodies involved in the autoimmune process or, on the other hand, at influencing the pathological process in a specific manner by producing immunotolerance. For blocking the antibodies, it is possible to use antibodies raised against the autoantibodies to OPG or to develop small molecules that block the interaction between the autoantibodies and OPG.

Such blocking or therapeutic antibodies are understood to be an immunoglobulin that specifically binds to, and is thereby defined as complementary with anti-OPG. The antibody can be monoclonal or polyclonal and can be prepared by techniques that are well known in the art such as immunisation of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a and IgG3, IgM, etc. Fragments thereof may include Fab, Fv and F(ab′)2, Fab′, and the like. In addition, aggregates, polymers and conjugates of immunoglobulins or their fragments can be used where appropriate so long as binding affinity for a particular polypeptide is maintained.

Alternatively, the OPG can be used in complete form or in the form of an adduct, phosphorylation product, partial peptide, peptide analogue or splice variant as a therapeutic agent for inducing immunotolerance or inducing blocking of the T-cell reactivity in antigen-presenting cells or T-cells by blocking or modulation of the antigen presentation.

Thus, in addition to providing the detection methods described above, there is provided a method of treating patients with osteoporosis, autoimmune disease and/or vascular disease or those at risk of developing such diseases which comprises:

(a) detecting the disease or propensity of developing the disease by screening a patient for autoantibody to OPG; and
(b) treating patients positive in the test of step (a) either with

(i) an inhibitor of osteoclastic bone resorption, such as Zoledronic acid, alendronic acid, risedronate disodium, calcitonin and salts and solvates thereof;

(ii) antibodies raised against the auto-antibodies that recognize OPG, or peptides, such as those derived from RANKL and small molecules which prevent binding of OPG antibodies to OPG or OPG fragments;

(iii) OPG or fragments thereof to restore immune tolerance to OPG, or

(iv) a combination of treatments (i)-(iii).

In a further aspect there is provided use of an antibody raised against an autoantibody to OPG for use in treating osteoporosis and/or related autoimmune or vascular conditions. In a further embodiment the present invention provides a method of treating osteoporosis and/or related autoimmune or vascular conditions, comprising the step of administering a therapeutically effective amount of an antibody raised against an autoantibody to OPG.

For the use or methods according to the present invention, the antibodies raised against the anti-OPG auto-antibody or antigenic fragment thereof may be presented as a pharmaceutical formulation, comprising the antibody together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active antibody in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active antibody may be in powder form which is constituted with a suitable vehicle, such as sterile, pyrogen-free water, before use.

An active antibody may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically function ing valve having the desired spray characteristics; advantageously the valve is of a metered type delivering a fixed volume, for example, 25 to 100 microlitres, upon each operation thereof.

As a further possibility an active antibody may be in the form of a solution or suspension for use in an atomizer or nebuliser whereby an accelerated airstream or ultrasonic agitation is employed to produce a fine droplet mist for inhalation. Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

In view of the inventor's findings, patients with autoimmune disease who may be considered at-risk of developing or having osteoporosis, should therefore be screened for OPG autoantibody according to the methods described herein. Those patients found to be positive for the autoantibody, and their family members, should be considered for treatment according to the methods described herein. The reliable immunological assay of this invention can be automated or developed as a finger stick screening test. Furthermore, this invention provides a kit for detecting OPG autoantibodies in a patient or in people at-risk of developing osteroporosis. The kit would contain a source of purified OPG or antigenic fragment thereof and optionally other reagents, such as RANK, RANKL and/or anti-OPG, any of these being optionally labelled.

As used herein, the term “kit” refers to any delivery system for delivering materials. In the context of reaction assays, such delivery systems include systems that allow for the storage, transport or delivery of reaction reagents (e.g., probes, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. Such contents may be delivered to the intended recipient together or separately. For example, a first container may contain a source of OPG for use in an assay, while a second container contains tags.

### DETAILED DESCRIPTION

The present invention will now be further described by way of example and with reference to the Figures which show:

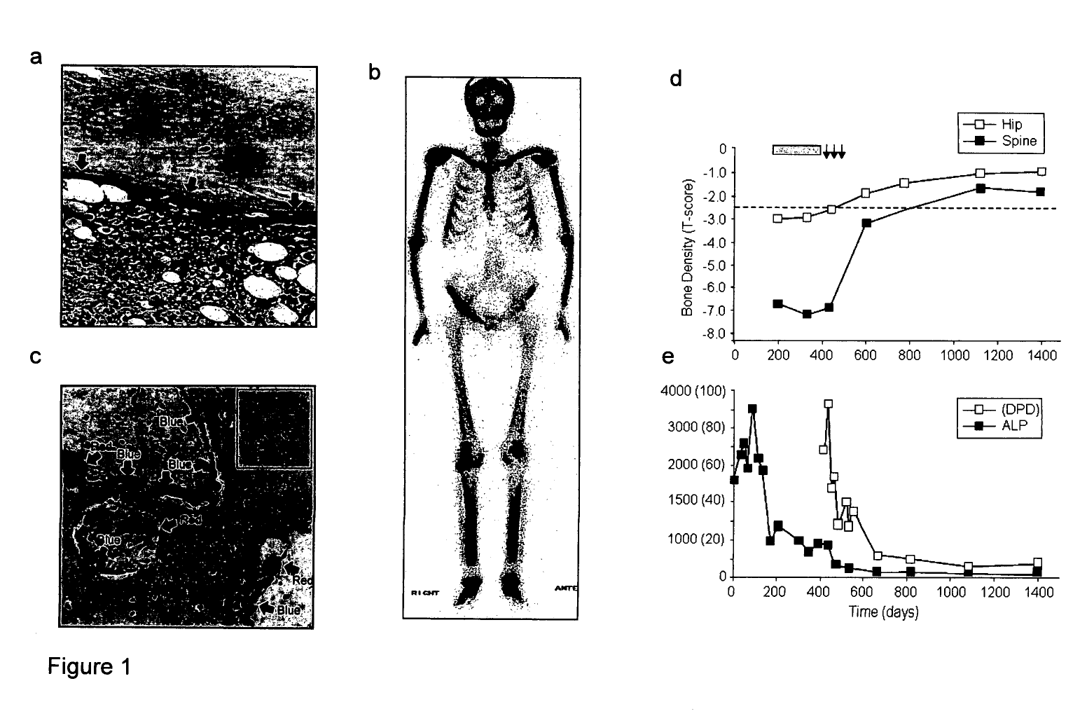

FIG. 1 shows clinical features of the subject and response to treatment Panel a. Photomicrograph of Transiliac bone biopsy stained with toluidine blue illustrating increased extent of osteoid seams, indicative of a mild mineralization defect. Panel b. Radionucleotide bone scan showing generalised increase in tracer uptake without evidence of focal lesions. Panel c. Photomicrograph of Transiliac bone biopsy stained with H&E showing marked increase in osteoclasts (red arrows) and osteoblasts (blue arrows). Woven bone is present throughout the biopsy as evidenced by the irregular cement lines. Panel d. Bone mineral density and Panel e, urinary deoxypyridinoline/creatinine ratio (DPD) and alkaline phosphatase (ALP) levels at presentation and in response to treatment. Bone density fell despite an extended period of treatment with a gluten free diet, calcium and vitamin D (grey bar), but BMD progressively increased following treatment with Zoledronic acid (arrows) (panel c). Serum ALP levels fell from over 3000 u/ml to about 1000 u/ml following treatment with a gluten free diet, calcium and vitamin D, but DPD remained grossly elevated. Treatment with Zoledronic acid (arrows) normalised both biochemical markers of bone turnover.

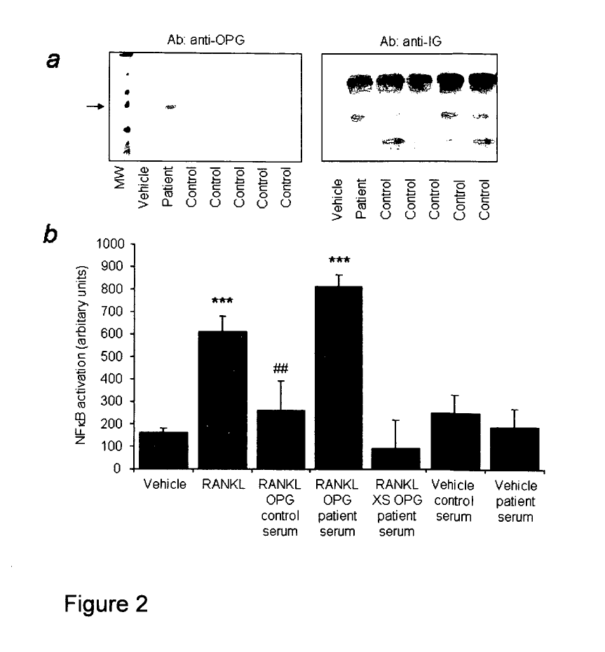

FIG. 2 shows the detection of neutralising autoantibodies to osteoprotegrin Panel a. The left panel shows the presence of autoantibodies directed against osteoprotegerin in the patient's serum, reflected by the detection of a 55 Kd band on western blot by an anti-OPG antibody following immunoprecipitation. Samples from controls failed to immunoprecipitate OPG under the same conditions. The top right panel is stained for immunoglobulin to confirm equal loading of the gel. The result shown is representative of three independent experiments. Panel b. Patient serum, but not control serum abrogates the inhibitory effect of 100 ng/ml OPG on RANKL stimulated NFκB activation in HEK 239 cells. Addition of 400 ng/ml OPG (column labelled XS OPG) overcame the inhibition. ** p<0.001 from vehicle; ## p<0.01 from RANKL and from RANKL+OPG+patient serum. The result is representative of three independent experiments.

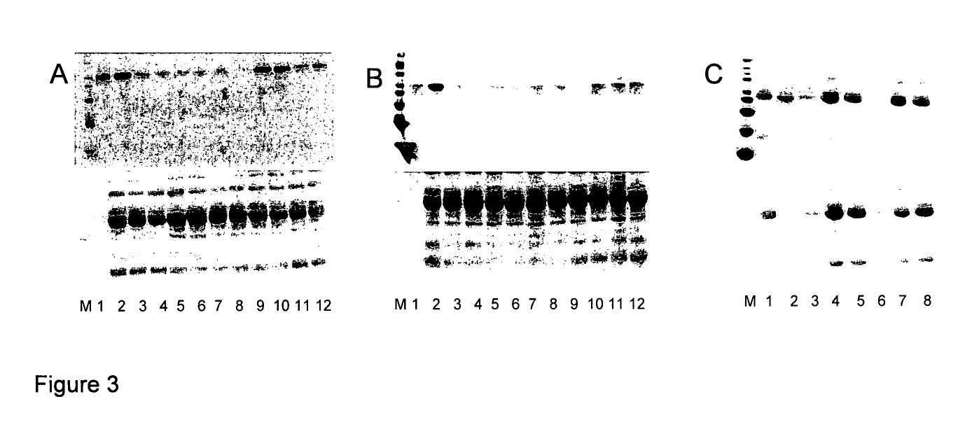

FIG. 3: (A) demonstrates the presence of autoantibodies to OPG in the patient's serum (lane 2) reflected by the detection of a strong band at 55 Kd on western blot by an anti-OPG antibody following immunoprecipitation. Lane I shows a negative control. Lanes 3-12 show results from patients 1-10 with celiac disease and evidence of OPG autoantibodies is present in lanes 9 and 10. The lower panel shows the western blot stained for immunoglobulin to show equal loading of the gel. (B) Lane 1 shows a negative control and lane 2 shows a positive control of the index patient's serum. Lanes 3-12 show patients 11-20 with celiac disease and evidence of OPG autoantibodies is present in lanes 10-12. Lower panel shows the western blot stained for immunoglobulin to show equal loading of the gel: (C) Lane 1 shows a positive control of the index patient's serum and lane 2 shows a negative control. Lanes 3-8 show patients 21-26 with celiac disease and evidence of OPG autoantibodies is present in lanes 4 and 7. Lower panel shows the western blot stained for immunoglobulin to show lanes were not equally loaded.

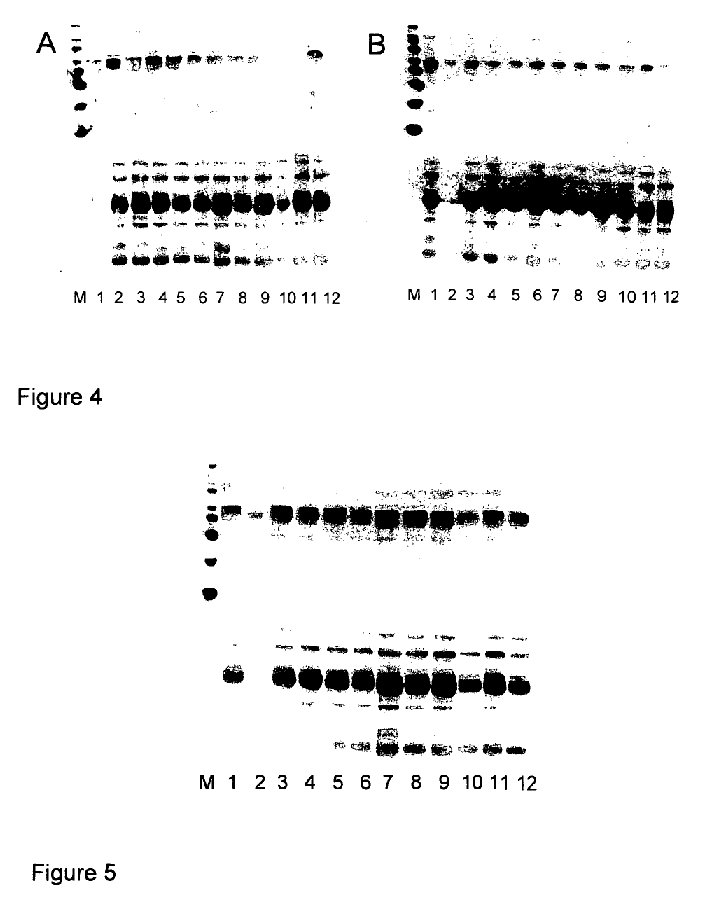

FIG. 4: (A) Lane 1 shows a negative control and lane 2 shows a positive control of the index patient's serum. Lanes 3-12 show results from patients 27-36 with celiac disease and evidence of OPG autoantibodies is present in lanes 4, 5, 6 and 12. The lower panel shows the western slot stained for immunoglobulin to show equal loading of the gel. (B) Lane 1 shows a positive control of the index patients serum and lane 2 shows a negative control. Lanes 3-12 show patients 37-48 with celiac disease and evidence of OPG autoantibodies is present in lanes 3, 6, 7 and 11. Lower panel shows western blot stained for Immunoglobulin to show equal loading of the gel.

FIG. 5: Lane 1 shows a positive control of the index patient's serum and lane 2 shows a negative control. Lanes 3-12 show results from patients 1-10 with idiopathic osteoporosis. Evidence of OPG autoantibodies is present in lanes 3, 4, 5, 7, 8, 9 and 11. The lower panel shows the western blot stained for immunoglobulin to show equal loading of the gel.

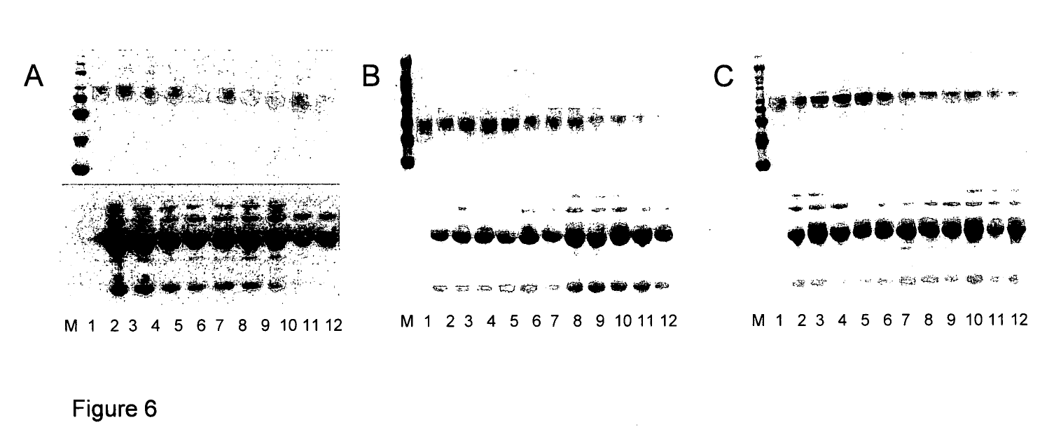

FIG. 6: (A) Lane 1 shows a negative control and lane 2 shows a positive control of the index patient's serum. Lanes 3-12 show patients 1-10 with autoimmune hypothyroidism and evidence of OPG autoantibodies is present in lane 2. The lower panel shows the western blot stained for immunoglobulin to show equal loading of the gel. (B) Lane 1 shows a negative control. Lanes 2-12 show patients 11-21 with autoimmune hypothyroidism and confirmed TRAB autoantibodies in their serum. Evidence of OPG autoantibodies is present in lanes 3, 4 and 5. The lower panel shows the western blot stained for immunoglobulin to show equal loading of the gel. (C) Lane 1 shows a negative control, Lanes 2-12 show patients 22-31 with autoimmune hypothyroidism and confirmed TRAB autoantibodies in their serum. Evidence of OPG autoantibodies is present in lanes 2, 3, 4, 5 and 6. The lower panel shows the western blot stained for immunoglobulin to show equal loading of the gel.

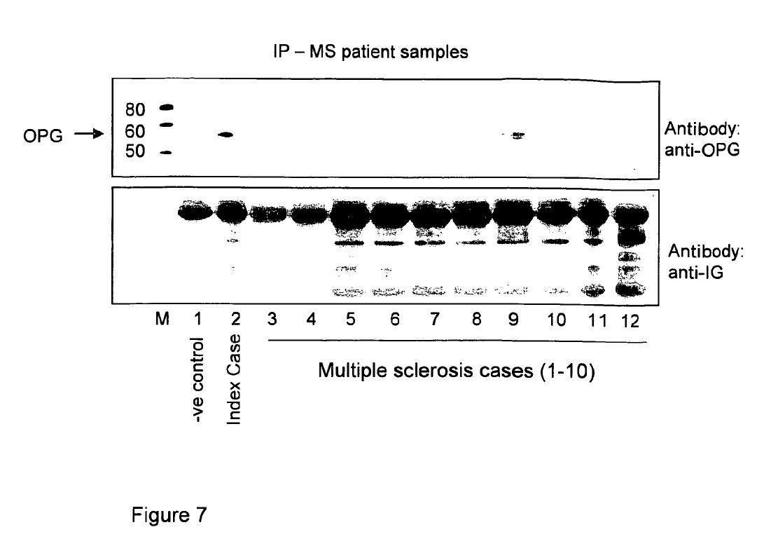

FIG. 7: Prevalence in multiple sclerosis: Representative immunoprecipitation assay from unselected patients with multiple sclerosis. OPG antibody is demonstrated in lane 9. The bottom panel shows the same blot probed with anti human IgG demonstrating equal loading. Overall prevalence in multiple sclerosis from preliminary cohort of 20 patients was 10%. Lane 1 is a negative control and lane 2 is a sample from the index patient.

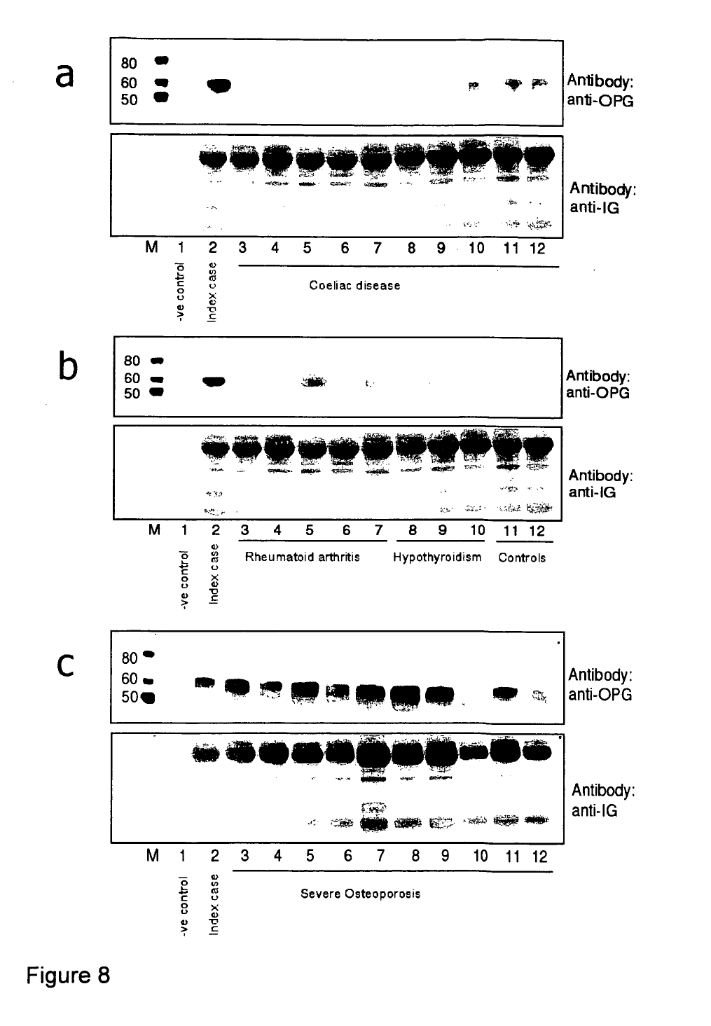

FIG. 8: Prevalence in further disease cohorts. A: Representative immunoprecipitation assay from unselected patients with coeliac disease. OPG antibody is demonstrated in lanes 10 to 12. The bottom panel shows the same blot probed with anti human IG demonstrating equal loading. Lane 1 is a negative control, lane 2 is the index patient. B: Representative immunoprecipitation assay from patients with rheumatoid arthritis (lanes 3 to 7) and hypothyroidism (lanes 8-10). Lanes 11-12 were healthy controls. Again Lane 1 is a negative control and lane 2 the index patient. C: Immunoprecipitation assay demonstrating presence of OPG antibody in unselected patients with severe osteoporosis (samples in lanes 3 to 12). The patients in lanes 6 & 8 also had treated hypothyroidism. Lane 1 is a negative control, lane 2 is the index patient.

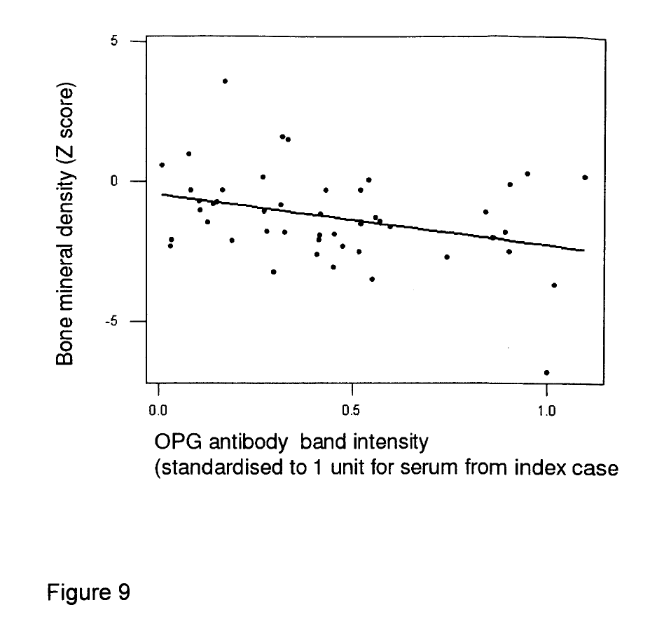

FIG. 9: Titre of antibody and bone mineral density. The intensity of the bands seen with severe osteoporosis appears more marked than in the autoimmune cohorts where typically osteoporosis was less severe. Though the immunoprecipitation assay is not truly quantifiable, standardising results for loading of serum and against the positive control suggests a modest, statistically significant, correlation between intensity of the OPG band and bone mineral density (r=−0.33, p=0.023).

### Case Report

A 40-year old Caucasian man presented following a low trauma fracture to his left clavicle which occurred whilst playing with his son. He had been previously fit and healthy, with no fracture history despite regularly playing rugby into his late 30's. Dual energy x-ray absorptiometry (DEXA) was performed and demonstrated low bone mineral density (BMD) with a T-Score of −6.6 at the spine, and −2.9 at the femoral neck. At this time routine investigations were carried out, showing an elevated alkaline phosphatase (ALP) of 2601 U/L (normal range 25-120 U/L) and this was shown to be of bony origin with isoenzyme studies. The patient also had an elevated serum phosphate of 2.36 mmol/L (normal range 0.8-1.4 mmol/L). Full blood count, urea and electrolytes, liver function tests, serum calcium, albumin levels and a short synacthen (ACTH) test were all normal at this time. Serum parathyroid hormone (PTH) level was low at 8 ng/L (normal range 10-65 ng/L), and the patients level of serum 25(OH)D was shown to be within the normal range at 35 ng/L (normal range 25-150 ng/L).

For a year prior to presentation the patient had noticed a lack of energy and intolerance to cold weather, and therefore was screened for hypothyroidism. His serum free thyroxine was shown to be <5 pmol/l (normal range 10-20 pmol/L) and his thyroid stimulating hormone (TSH) level was high at >65 mU/L (normal range 2-5 mU/L). Anti-thyroid peroxidase autoantibodies were detected at a level of 243 u/mL (normal range 0-82 u/ml), but tests for TSH receptor blocking autoantibodies were all negative. Serum testosterone and gonadotrophins were normal, and the patient was commenced on L-thyroxine 100 mcg/day for treatment of confirmed hypothyroidism.

ALP levels continued to rise to 3539 U/L and the patient developed hypercalcaemia with a serum calcium level of 2.8 mmol/L (normal range 2.1-2.6 mmol/L). At this time the level of serum PTH and 1.25(OH)D were undetectable. The urinary calcium/creatinine ratio was elevated to 4.78 (normal <0.5) as is consistent with a resorptive mechanism of hypercalcaemia, which was then resolved with intravenous rehydration. Extensive investigations were performed to exclude occult malignancy including bone marrow aspirate, CT abdomen and thorax and myeloma screen but the results were unremarkable and a radionuclide bone scan showed diffusely increased tracer uptake throughout the skeleton without focal lesions (FIGS. 1a and 1b) Transilic bone biopsy showed high bone turnover with dramatic increase in osteoclast and osteoblast activity with woven bone (FIG. 1c). There was a mild increase in extent of osteoid seams, but osteoid thickness was normal (FIG. 1d).

Titres of transglutaminase IgA antibodies were raised at 101 u/L (normal range 5-30 u/L) and celiac disease was confirmed by a small bowel biopsy. Treatment was started for this with a strict gluten-free diet, ergocalciferol 10,000 units/day and calcium supplementation of 1 g/day. At follow-up 6 months later the patient had an ALP level of 1000 U/L however his osteoporosis had worsened producing a T-Score of −7.1 on repeat DEXA. The patient suffered a low trauma fracture of his left humerus, and lost 6 cm in height due to multiple vertebral fractures. At this time repeat duodenal biopsy demonstrated normalisation of bowel histology and the patients serum 1.25(OH)D levels had were within the normal range.

Serum osteoprotegerin (OPG) levels were measured by ELISA (Biomedica, Oxford Biosystems, Oxford UK) on two occasions a month apart, demonstrating levels of 0.78 μmol/L and 0.47 μmol/L respectively (normal range 0.14-130 μmol/L) At the same times serum total RANKL levels were 0.152 and 0.143 nmol/L (normal range 0-10 nmol/L) as measured by ELISA (Apotech, Epalinges, Switzerland).

In light of his continued clinical deterioration the patient was treated with 3 infusions of 4 mg zoledronic acid over a 3-month period. Following the initial dose serum calcium levels decreased to 1.91 mmol/L and this was accompanied by an elevation in PTH to 163 ng/L, and 1.25(OH)₂D₃ to 992 pmol/L (normal range 15-150 pmol/L). Following completion of the treatment regime all serum derangements had returned to within normal ranges. At follow-up 42 months after the initial presentation the patient had not experienced any further fractures; his height had remained stable, and repeat DEXA demonstrated an improvement in T-Score to −1.7.

### EXAMPLE 1

### Methods

### Immunoprecipitation Assay for OPG

Non fasting serum samples were obtained from the patient on several occasions throughout his illness, and from 10 healthy age matched male controls. Further screening was carried out in 20 samples from patients with celiac disease and 14 with primary hypothyroidism. Protein content was measured using the bicinchonic acid assay (Pierce). For the immunoprecipitation assays, we incubated patient or control serum at 1 in 100 dilution with 12.5 ng recombinant human OPG (R&D systems) and protein G coated agarose beads (Calbiochem) which had been pre-incubated with 5% albumin to reduce non-specific binding. Following incubation for 1 hour at 37° C., the protein G agarose beads were spun down, and washed 5 times with PBS. After the final wash the beads were spun down, resuspended in 30 μl reducing sample buffer and incubated at 90° C. for 5 minutes. After allowing the samples to cool down, the beads were spun down and the supernatant was loaded onto a 12% polyacrylamide gel (Biorad Criterion) and electrophoresis performed at 200 V for 60 min. After electrophoresis, the proteins were transferred to a charged nylon membrane by Western blotting and the membrane probed with a mouse monoclonal antibody to human OPG (Abcam), with detection by a peroxidase conjugated donkey anti-mouse antibody (Jackson) at a 1/5000 dilution. Loading of the samples was assessed by probing the blot with peroxidase conjugated goat anti-human antibody (Jackson) at a 1/5000 dilution. Immunolabeled bands were detected using chemiluminescence (Supersignal Pierce) on a Syngene GeneGnome imager.

### RANK Signaling Assay

We studied the effects of the patient's serum and control sera on RANKL induced NFκB activation using HEK293 cells which had been stably transfected with a NFκB-responsive luciferase reporter vector (Cambridge Biosciences). The cells were maintained in DMEM supplemented with 10% fetal calf serum and Hygromycin (Roche), but were serum starved for 2 hours prior to RANKL stimulation and maintained thereafter in DMEM with 2% cytokine deplete TCH supplement (MP biomedicals). Serum samples were purified using protein G spin columns (Pierce) according to the manufacturer's instructions to avoid any non specific serum effect. Human recombinant RANKL (Prosckelia, 100 ng/ml) was incubated for 1 h at 37° C. with either OPG alone (R&D systems, 100 to 400 ng/ml), OPG with serum (1:40), or vehicle. After serum starving cells were stimulated with human RANKL preparations for 4 hours. Next the cells were lysed and the lysates analysed for luciferase activity using the Steady Glo reagent (Promega) using a Biotek Synergy HT platereader. All reporter assays were run with 5 replicates in 96 well plate containing 5×10⁴ cells per well.

### Discussion

The patient described here presented at the age of 40 with severe, high turnover osteoporosis associated with the development of celiac and thyroid disease. We have demonstrated novel antibodies to OPG and shown that the patient's serum, but not control serum, reversed the inhibitory effects of OPG on RANKL induced RANK signaling in vitro. This is the first report of a clinical illness due to the spontaneous development of antibodies to OPG. We are aware of the fact that therapeutic administration of a recombinant OPG construct (AMGN-0007) in one patient resulted in the development of antibodies to this product. However this was not associated with a clinical illness. The mechanisms by which OPG antibodies developed in this patient remain unclear, but auto-antibodies to circulating proteins have previously been described in patients with autoimmune disease (10).

Bone density measurements had not been performed in our patient prior to his presenting illness, but an inherited form of osteoporosis is excluded by the lack of a positive family history and the fact that he had never sustained a fracture despite leading a highly active lifestyle that included playing rugby. It is also extremely unlikely that either the celiac disease or hypothyroidism contributed substantially to this illness. Celiac disease can be associated with osteoporosis and osteomalacia due to malabsorption of calcium, vitamin D and other nutrients. The severity of the osteoporosis and high bone turnover in the presence of only a mild mineralization defect is out of keeping with what one would expect in celiac disease and treatment with a gluten-free diet, calcium supplements and vitamin D failed to influence progression of the osteoporosis substantially. We also considered the possibility that the autoimmune thyroid disease might have contributed to the osteoporosis in light of a previous report which suggested that TSH inhibits bone remodelling (11). To investigate this possibility we screened for the presence of TSH receptor blocking antibodies which conceivably could have blocked an inhibitory effect of the raised TSH levels on bone turnover but these were negative. In this regard it should be noted that when bone abnormalities are seen in adult hypothyroidism, it is typically a low bone turnover state which is in complete contrast to the clinical picture described here. In view of this it seems extremely unlikely that the osteoporosis was in any way related to the hypothyroidism, which is in keeping with the results of other studies which have indicated that it is thyroid hormone, rather than TSH which is primarily responsible for the regulation of bone remodelling (12).

The florid bone disease in our patient fits well with the reported phenotype seen in inherited OPG deficiency in mice (4) and humans (2;3) and since we have found no other sufficient explanation it seems almost certain that the development of antibodies to OPG is the cause of his illness. We subsequently showed the presence of anti-OPG antibodies in 5 out of 20 additional celiac patients, indicating that these antibodies may be fairly common in these auto-immune patients. The antibody levels appeared to be much lower in these than in the original patient however, which one would expect would lead to a less severe effect on bone turnover. The severity of the bone disease and clinical deterioration despite treatment of the celiac disease and supplementation with calcium and vitamin D is matched only by the remarkable recovery that occurred in response to treatment with the potent inhibitor of osteoclastic bone resorption, Zoledronic acid. Our studies demonstrate that autoantibodies to OPG can develop spontaneously in the context of autoimmune disease, raising the possibility that antibodies to OPG might contribute to the pathogenesis of raised bone turnover and osteoporosis in other inflammatory diseases.

In addition to the identification of autoantibodies to OPG in Celiac patients, there may be a correlation with bone density also and as such, bone density measurements may also be carried out in conjunction with autoantibody detection to OPG in Celiac patients. Indeed, the present inventors have observed that some Celiac patients with OPG autoantibodies do have lower bone mineral density.

Finally, the present inventors also looked at a conventional serum RANKL assay. This experiment involved performing an ELISA for serum RANKL in a patient using an assay which detects the free fraction of serum RANKL (Biomedica, Vienna, Austria) according to the manufacturers instructions. The samples were assayed undiluted and at dilutions in water. The results (see Table 1) showed an increase in RANKL as the samples were diluted, consistent with the presence of an antibody that was interfering with the binding of RANKL to OPG which in this assay is used to “capture” the RANKL. This perhaps suggests that currently available serum RANKL assays are being affected by OPG autoantibodies.

### EXAMPLE 2

### Methods

Serum samples were obtained from the following cohorts:
The patient himself; several samples were obtained at various points through his illness.
10 age-matched healthy male control patients (+/−10 years of index patients age)
46 patients with celiac disease
31 patients with autoimmune hypothyroidism
10 patients with idiopathic severe osteoporosis
10 patients with rheumatoid arthritis
20 patients with multiple sclerosis

All samples were obtained non-fasting and protein count was measured using the bicinchonic acid assay (Pierce). Collected serum samples were stored at 4° C. and purified within one week of collection. The purification process followed the standard protocol for protein G spin columns (Pierce).

### Immunoprecipitation Assay for OPG

For the immunoprecipitation assays, protein G coated agarose beads (Calbiochem) were pre-incubated for 1 hour at 37.5° C. with 5% human recombinant albumin to reduce non-specific binding. Beads were then incubated for 1 hour at 37.5° C. with serum samples at a 1/100 dilution with 12.5 ng of homodimeric recombinant human OPG (R&D systems). Beads were then washed five times with pre-warmed PBS, suspended in 30 ml of reducing sample buffer and incubated at 90° C. for 5 minutes. Following brief centrifugation the supernatant was loaded into a 12% polyacylamide gel (Biorad Criterion) which underwent electrophoresis at 200V for 45 minutes. Following western blotting the membrane was probed with a mouse monoclonal antibody to human OPG (Abcam), with detection by a peroxidase conjugated donkey anti-mouse antibody (Jackson) at a 1/5000 dilution. Immunolabelled bands were detected using chemiluminescence (Supersignal Pierce) on a GeneGnome imager (Syngene). Equal loading of samples was confirmed by stripping each blot, and probing for total immunoglobulin with peroxidase conjugated goat anti-human antibody (Jackson) at a 1/5000 dilution.

During experimentation, the index patient's serum was used to provide a positive control band, and a negative control band was provided by using rabbit anti-actin antibody.

### RANK Signalling Assay

The effects of immunoglobulin purified from the patient's serum and control sera on RANKL induced NFκB activation were studied using stably transfected HEK293 cells with a NFκB-responsive luciferase reporter vector (Cambridge Biosciences). Cells were maintained in DMEM supplemented with 10% fetal calf serum and Hygromycin (Roche). Cells were starved for 2 hours prior to stimulation and maintained thereafter in DMEM with 2% cytokine deplete TCH supplement (MP Biomedicals). All assays were run with five replicated in a 96 well plate, containing 5×10⁴ cells per well. The cells were stimulated with 100 ng/ml human recombinant RANKL for 1 hour at 37° C. in the presence of 100-400 ng/ml OPG (R&D systems) and in the presence or absence of immunoglobulins at a 1/40 dilution. Following stimulation cells were lysed and analyzed for luciferase activity using Steady Glo reagent (Promega) with a Biotek Synergy HT plate reader.

### Statistical Analysis

Bone density data was obtained for patients having undergone previous DEXA scanning as part of their clinical management. Each disease cohort was split into two groups; patients with evidence of OPG autoantibodies in their serum, and patients without evidence of OPG autoantibodies in their serum. Mean BMD data for these two cohorts were statistically compared using a t-test to establish whether a significant difference was present between the two groups. A confidence interval of 95% was used and significance was achieved if p<0.05.

### Results

The patient's serum immunoprecipitated OPG; shown by the appearance of a strong band at 55 Kd on the western blot (FIG. 2). Serum from 10 healthy male control patients yielded negative results. The IP was repeated in the presence of gliadin in order to see whether OPG autoantibodies cross-reacted with the protein causing an immune reaction in patients with celiac disease. It was shown that the presence of gliadin had no effect on the ability of the patient's serum to immunoprecipitate OPG.

BMD data was available for 36 patients from this cohort of 46 patients with celiac disease. The mean BMD T-Score values in this cohort were −1.116 for patients without OPG autoantibodies and −2.237 for those patients with evidence of OPG autoantibodies. This difference was statistically significant (p=0.011; significance if p≦0.05). BMD T-scores are standardised against young healthy controls, whilst BMD Z-scores are standardised against age matched controls. Mean age of patients with evidence of OPG autoantibodies in their serum was 67.5 years, whilst mean age of patients without evidence of OPG autoantibodies was 54.2 years. Patients without OPG autoantibodies had a mean spine Z-Score of −0.812, whilst patients with OPG autoantibodies had a mean spine Z-Score of −0.431. This difference was not statistically significant (p=0.254).

Evidence of OPG autoantibodies were also detected in 7 out of 10 patients with severe idiopathic osteoporosis and 4 out of 31 patients with autoimmune hypothyroidism (FIGS. 5 and 6). Evidence of OPG antibodies was also seen in 2 out of 20 patients with multiple sclerosis (FIG. 7) and 2 out of 10 patients with rheumatoid arthritis (FIG. 8b). The mean BMD T-scores for the small cohort of patients with severe idiopathic osteoporosis were −3.6 for patients without OPG autoantibodies and −3.4 for patients with evidence of OPG autoantibodies in their serum. This difference was not significant (p=0.36). BMD Z-scores were not available for this group of patients. Unfortunately, BMD data for the cohort of patients with autoimmune hypothyroidism were not available.

Addition of RANKL to the cell line used for the signalling assay caused significant activation of reporter gene expression (FIG. 2b). This was shown to be inhibited by an equal concentration of human recombinant OPG in the presence of the immunoglobulin fraction of control serum, however in the presence of the immunoglobulin fraction of the patient's serum the inhibitory effect of OPG was lost. Addition of OPG at higher concentration overcame this inhibition (400 ng/ml). In the absence of RANKL addition of either patient immunoglobulin, or control immunoglobulin was shown to have no significant effect on reporter gene expression. The RANK signalling assay demonstrated that the addition of the patient's purified serum to OPG inhibited RANKL caused loss of the inhibitory effect of OPG in vitro.

### Discussion

The patient discussed in this study presented in his 40s with low trauma fracture to his clavicle, and was shown to have developed severe high turnover osteoporosis, associated with celiac disease and autoimmune hypothyroidism. The patient was shown to have circulating neutralising autoantibodies to OPG; the patient's serum was shown to inhibit the effect of OPG on RANKL-induced NFκB signalling in vitro. Serum taken from age-matched healthy control patients did not have this effect.

This is the first report of a bone disease occurring in association with the spontaneous development of autoantibodies to OPG. The mechanism by which OPG autoantibodies developed in this patient remains unclear, however autoantibodies to other circulating proteins have been reported to develop in patients with autoimmune disease [12], and presumably OPG had become the target of an endogenous immune response in this patient.

Other possible diagnoses for this patient have been considered. A congenital inherited form of osteoporosis was excluded early as a possible cause, due to the late onset of the disease, and the fact that the patient had regularly undertaken high contact sports such as rugby into his late 30s, yet had no fracture history. Osteoporosis and osteomalacia are accepted complications of celiac disease due to deficiency of vitamin D and calcium [2,3]. These options were discarded due to the severity of osteoporosis, quick onset and extremely high level of bone turnover, and also the failure of usual treatments for celiac disease to improve BMD, including a strict gluten-free diet and calcium and vitamin D supplements. A diagnosis of fibrogenesis imperfecta ossium (FIO) was also considered as patients usually present in adulthood with fragility fractures. However the clinical features of this patient were distinctly different to patients with FIO, which is characterised by trabecular thickening on x-ray, accumulation of thick osteoid on bone biopsy and a poor response to treatment [13].

This study has also demonstrated the presence of OPG autoantibodies in small patient cohorts of patients with autoimmune hypothyroidism and severe idiopathic osteoporosis. The patients identified to have OPG autoantibodies were shown to have a lower mean BMD value than those without OPG antibodies and when comparing the presence or absence of OPG autoantibodies against BMD T-Scores within the cohort of patients with celiac disease a significant difference was identified. BMD is a complex trait which is influenced by both genetic and environmental factors, and as such data for other risk factors that may have affected BMD scores were sought. The age of all patients within the celiac disease cohort was known and a mean age difference of over 10 years was identified (mean age of patients with OPG autoantibodies=67.5 years, mean age of patients without OPG autoantibodies=54.2 years). Following this finding, a comparison of BMD Z-Scores and presence or absence of OPG autoantibodies was made, as BMD Z-Scores correct against age-matched healthy controls, whereas BMD T-Scores are corrected against young healthy controls [15]. This result was not statistically significant (p=0.254), however this is not surprising given the large difference in age between the two small cohorts.

It is extremely tempting to speculate that the OPG antibodies contribute to the pathogenesis of osteoporosis in autoimmune disease, and those patients with OPG autoantibodies are those that suffer with worse osteoporosis. It is also exciting to establish the presence of OPG autoantibodies in patients with unexplained severe osteoporosis. No healthy controls showed evidence of OPG autoantibodies, and therefore it is possible that the presence of OPG autoantibodies in a patient's serum could act as a distinct marker for the future development of severe osteoporosis. This would allow clinicians to establish which of their patients are at a high risk of developing even idiopathic osteoporosis, allowing close monitoring and earlier intervention to reduce environmental and lifestyle risk factors. However, it is important for researchers to bear in mind that BMD is an extremely complex trait which is influenced by both genetic and environmental factors throughout a patient's life, and therefore the results obtained in this study should be interpreted with some caution, particularly with the small cohort sizes studied. Unfortunately robust analysis of the data when corrected for age differences between cohorts of patients with celiac disease is not statistically significant, and further research will be necessary to establish whether a truly significant relationship exists between OPG autoantibodies and development of low BMD.

### REFERENCES CITED IN EXAMPLE 1

(1) Khosla S. Minireview: The OPG/RANKL/RANK System. Endocrinology 2001; 142(12):5050-5055.
(2) Whyte M P, Obrecht S E, Finnegan P M et al. Osteoprotegerin deficiency and juvenile Paget's disease. N Engl J Med 2002; 347(3):175-184.
(3) Chong B, Hegde M, Fawkner M et al. Idiopathic hyperphosphatasia and TNFRSF11B mutations: relationships between phenotype and genotype. J Bone Miner Res 2003; 18(12):2095-2104.
(4) Bucay N, Sarosi I, Dunstan C R et al. osteoprotegerin-deficient mice develop early onset osteoporosis and arterial calcification. Genes & Development 1998; 12(9):1260-1268.
(5) Beyens G, Daroszewska A, De Freitas F et al. Identification of sex specific effects of TNFRSF11B polymorphisms on the risk of Paget's disease of bone. Calcif Tiss Int 2007; 80(Supplement 1):S42.
(6) Daroszewska A, Hocking L J, McGuigan F E A et al. Susceptibility to Paget's disease of bone is influenced by a common polymorphic variant of Osteoprotegerin. Journal of Bone & Mineral Research 2004; 19(9):1506-1511.
(7) Styrkarsdottir U, Halldorsson B V, Gretarsdottir S et al. Multiple Genetic Loci for Bone Mineral Density and Fractures. N Engl J Med 2008.
(8) Richards J B, Rivadeneira F, Inouye M et al. Bone mineral density, osteoporosis, and osteoporotic fractures: a genome-wide association study. Lancet 2008; 371(9623):1505-1512.
(9) Sobacchi C, Frattini A, Guerrini M M et al. Osteoclast-poor human osteopetrosis due to mutations in the gene encoding RANKL. Nat Genet 2007; 39(8):960-962.
(10) O'Neill S G, Isenberg D A, Rahman A. Could antibodies to C-reactive protein link inflammation and cardiovascular disease in patients with systemic lupus erythematosus? Annals of the Rheumatic Diseases 2007; 66(8):989-991.
(11) Abe E, Marians R C, Yu W et al. TSH is a negative regulator of skeletal remodeling. Cell 2003; 115(2):151-162.
(12) Bassett J H, O'Shea P J, Sriskantharajah S et al. Thyroid hormone excess rather than thyrotropin deficiency induces osteoporosis in hyperthyroidism. Mol Endocrinol 2007; 21(5):1095-1107.

### REFERENCES CITED IN EXAMPLE 2

(1) Riggs, B. L., Melton, L. J. The worldwide problem of osteoporosis: Insights afforded by epidemiology. Bone. 1995; 17(5): S505-S511
(2) Bianchi, M. L., Bardella, M. T. Bone and celiac disease. Calcified Tissue International. 2002; 71(6): 465-471
(3) Meyer, D., Stavropolous, S., Diamond, B., Shane, E., Green, P. H. Osteoporosis in a north American adult population with celiac disease. American Journal of Gastroenterology. 2001; 96(1): 112-119
(4) Pazianas, M., Butcher, G. P., Subhani, J. M. et al. Calcium absorption and bone mineral density in celiac after long term treatment with gluten-free diet and adequate calcium intake. Osteoporosis International. 2005; 16(1): 56-63
(5) Khosla, S. Mini-review: The OPG/RANKL/RANK System. Endocrinology. 2001; 142(12): 5050-5055
(6) Feige, U. Osteoprotegerin. Annals of the Rheumatic Diseases. 2001; 60: 81-84
(7) Sobacchi, C., Frattini, A., Guerrini M. M., et al. Osteoclast-poor human osteopetrosis due to mutations in the gene encoding RANKL. Nature Genetics. 2007; 39(8): 960-962
(8) Whyte, M. P., Obrecht, S. E., Finnegan, P. M., et al. Osteoprotegerin deficiency and juvenile Paget's disease. New England Journal of Medicine. 2002; 347(3): 175-184
(9) Chong, B., Hegde, M., Fawkner, M., et al. Idiopathic hyperphosphatasia and TNFRSF11B mutations: relationships between phenotype and genotype. Journal of Bone and Mineral Research. 2003; 18(12): 2095-2104
(10) Bucay N, Sarosi I, Dunstan C R et al. osteoprotegerin-deficient mice develop early onset osteoporosis and arterial calcification. Genes & Development 1998; 12(9): 1260-1268
(11) McClung, M. R., Lewiecki, E. M., Cohen, S. B., et al. Denosumab in postmenopausal women with low bone mineral density. New England Journal of Medicine. 2006; 354(8): 821-831
(12) O'Neill, S. G., Isenberg, D. A., Rahman, A. Could antibodies to C-reactive protein link inflammation and cardiovascular disease in patients with systemic lupus erythematosus? Annals of the Rheumatic Diseases. 2007; 66(8): 989-991
(13) Carr, A. J., Smith, R., Athanasou, N., Woods, C. G. Fibrogenesis imperfecta ossium. Journal of Bone and Joint Surgery (British Volume). 1995; 77(5): 820-829
(14) Nakachi, K., Swift, G., Wilmot, D., et al. Antibodies to tissue transglutaminase: comparison of ELISA and immunoprecipitation assay in the presence and in the absence of calcium ions. Clinica Chimica Acta. 2001; 304(1): 75-84
(15) Shepherd, J. A., Blake, G. M. T-Scores and Z-Scores. Journal of Clinical Densitometry. 2007; 10(4): 349-350

TABLE 1

| | | | RANKL Amplification | | | | |
|---|---|---|---|---|---|---|---|
| | | | pmol/L | | | | |
| | | | | ¼ | ¼ dil | ⅛ dil | 1/16 dil |
| | | sRANKL | | dil in | in | in | in |
| Patient X | OPG | (total) | neat | water | diluent | diluent | diluent |
| 08.06.05 | 0.78 | 152 | 0.02 | 0.23 | 0.4 | 1.06 | 1.09 |
| 16.06.05 | 0.81 | 226 | 0.05 | 0.5 | 0.8 | 0.9 | 1.74 |
| 27.06.05 | 0.88 | 213 | 0.01 | 0.39 | 0.5 | 0.34 | 0.19 |
| 07.07.05 | 0.47 | 143 | 0.05 | 0.19 | 1.3 | 1.54 | 2.77 |
| 12.09.05 | 2.06 | 135 | 0.05 | 0.86 | 1.48 | 2.26 | 0.99 |
| 01.09.08 | 2.84 | 181 | 0.02 | < | | | |

TABLE 2

| | | | RANKL Amplification | | |
|---|---|---|---|---|---|
| | | | method (pmol/L) | | |
| | | Old RANKL | | ¼ dil | ⅛ dil |
| | OPG | Method | Neat | in diluent | in diluent |
| Patient SG | | | | 0.74 | 2.51 |
| PMS4 | 2.0 | 3.80 | 0.01 | 0.26 | |
| SG | 2.5 | 2.44 | 0.17 | 0.62 | |
| JB | 2.6 | 1.41 | 0.53 | 0.61 | |
| JS | 2.5 | 0.27 | 0.13 | 0.27 | |
| LSC | 4.0 | 0.22 | 0.09 | 0.56 | |
| JG | 4.1 | 0.14 | 0.02 | 0.28 | |
| PMS1 | 3.6 | 0.26 | 0.19 | 0.52 | |
| MS2 | 2.8 | 0.26 | 0.55 | 0.14 | |
| PMS8 | 2.5 | 0.42 | 0.07 | 0.55 | |
| PMS5 | 2.5 | 0.37 | <0.01 | 0.46 | |
| MS3 | 2.3 | 0.23 | <0.01 | 0.18 | |
| MS5 | 2.2 | 0.20 | 0.04 | 0.08 | |
| PMG2 | 2.1 | 0.61 | 0.15 | 0.34 | |
| PMG4 | 2.1 | 0.48 | 0.08 | 0.50 | |

1. A method for detecting autoantibodies to osteoprotegerin (OPG), the method comprising the step of providing a biological sample from a subject with, or at risk of developing osteoporosis and detecting whether or not any antibodies against osteoprotegerin (OPG) are present in the biological sample.

2. The method according to claim 1 for aiding the diagnosis of osteoporosis or susceptibility to osteoporosis in autoimmune diseases.

3. The method according to claim 1 for aiding the diagnosis of susceptibility to vascular complications such as, for example, ischaemic heart disease, stroke and peripheral vascular disease, in patients with autoimmune disease.

4. A method for detecting autoantibodies to osteoprotegerin (OPG), the method comprising the step of providing a biological sample from a subject with, or at risk of developing autoimmune and/or vascular disease and detecting whether or not any antibodies against osteoprotegerin (OPG) are present in the biological sample.

5. The method of claim 1, wherein the sample is a sample of blood, serum, plasma, urine, saliva or leukocytes.

6. The method of claim 1, wherein the detection of said autoantibodies in the biological sample is by an immunodiagnostic method.

7-13. (canceled)

14. The method of claim 4, wherein the sample is a sample of blood, serum, plasma, urine, saliva or leukocytes.

15. The method of claim 4, wherein the detection of said autoantibodies in the biological sample is by an immunodiagnostic method.

16. The method according to claim 6, wherein the immunodiagnostic method is a radioimmunoassay, ELISA competitive binding assay or sandwich assay.

17. The method of claim 15, wherein the sample is a sample of blood, serum, plasma, urine, saliva or leukocytes.

18. A method of treating patients with osteoporosis, autoimmune disease and/or vascular disease or those at risk of developing such diseases which comprises:
(a) detecting said disease or a risk of developing the disease by screening a patient for an autoantibody to OPG; and
(b) treating patients positive in the test of step (a) either with
(i) an inhibitor of osteoclastic bone resorption, such as Zoledronic acid, alendronic acid, risedronate disodium, calcitonin and salts and solvates thereof;
(ii) antibodies raised against the auto-antibodies that recognize OPG, or peptides, such as those derived from RANKL and small molecules which prevent binding of OPG antibodies to OPG or OPG fragments;
(iii) OPG or fragments thereof to restore immune tolerance to OPG, or
(iv) a combination of treatments (i)-(iii).

19. The method according to claim 18, wherein the inhibitor of osteoclastic bone resorption is Zoledronic acid, alendronic acid, Risedronate disodium, calcitonin and salts and solvates thereof.

20. A method of treating osteoporosis and/or related autoimmune and/or vascular conditions, said method comprising the step of administering a therapeutically effective amount of an antibody raised against an autoantibody to OPG.

21. A kit for detecting OPG autoantibodies in a patient or in people at-risk of developing osteoporosis, autoimmune and/or vascular conditions comprising a source of purified OPG or antigenic fragment thereof and optionally other reagents, such as RANK, RANKL and/or anti-OPG, any of these being optionally labelled.

22. The method of claim 2, wherein the autoimmune disease or diseases is/are selected from the group consisting of celiac disease; rheumatoid arthritis; inflammatory bowel disease; autoimmune hypothyroidism; severe idiopathic osteoporosis; multiple sclerosis; SLE; scleroderma; and connective tissue disease.

23. The method of claim 3, wherein the autoimmune disease or diseases is/are selected from the group consisting of celiac disease; rheumatoid arthritis; inflammatory bowel disease; autoimmune hypothyroidism; severe idiopathic osteoporosis; multiple sclerosis; SLE; scleroderma; and connective tissue disease.

24. The method of claim 4, wherein the autoimmune disease or diseases is/are selected from the group consisting of celiac disease; rheumatoid arthritis; inflammatory bowel disease; autoimmune hypothyroidism; severe idiopathic osteoporosis; multiple sclerosis; SLE; scleroderma; and connective tissue disease.

25. The method of claim 18, wherein the autoimmune disease or diseases is/are selected from the group consisting of celiac disease; rheumatoid arthritis; inflammatory bowel disease; autoimmune hypothyroidism; severe idiopathic osteoporosis; multiple sclerosis; SLE; scleroderma; and connective tissue disease.

26. The method of claim 20, wherein the autoimmune condition is selected from the group consisting of celiac disease; rheumatoid arthritis; inflammatory bowel disease; autoimmune hypothyroidism; severe idiopathic osteoporosis; multiple sclerosis; SLE; scleroderma; and connective tissue disease.
